(19) United States
(12) Patent Application Publication
(10) Pub. No.: US 2026/0114752 A1
(43) Pub. Date: Apr. 30, 2026

(54) EQUINE MOTION SYSTEM SENSOR

(71) Applicant: Garmin International, Inc., Olathe, KS (US)

(72) Inventor: Christopher R. Koszarsky, Cary, NC (US)

(21) Appl. No.: 19/367,932

(22) Filed: Oct. 24, 2025

Related U.S. Application Data
(60) Provisional application No. 63/712,083, filed on Oct. 25, 2024.

(51) Int. Cl. A61B 5/11; A01K 29/00; A61B 5/00; G01P 15/18; G01S 19/13; H01Q 7/00

(52) U.S. Cl. CPC A61B 5/112; A01K 29/005; A61B 5/6813; A61B 5/6831; G01P 15/18; G01S 19/13; H01Q 7/00; A61B 2503/40; A61B 2562/0219

(57) ABSTRACT

An equine sensor system can comprise an equine sensor and a computing device. The equine sensor can include a strap removably coupled to a tail of an equine, an accelerometer configured to collect accelerometer data, and a transmitter coupled to the accelerometer configured to transmit the accelerometer data. The computing device can include a memory, a receiver configured to receive the accelerometer data, and a processor coupled to the memory and the receiver, wherein the processor is configured to determine a stride length of the equine based on the accelerometer data and positional data.

### RELATED APPLICATIONS

This application claims priority to U.S. provisional application 63/712,083 filed Oct. 25, 2024, the contents of which are hereby incorporated by reference herein for all purposes.

### TECHNICAL FIELD

The present disclosure relates generally to an equine sensor system.

### BACKGROUND

Equine health management can include monitoring and assessing movement and biomechanical performance of an equine in an attempt to identify lameness, limb issues, and gait abnormalities.

### BRIEF DESCRIPTION OF THE DRAWINGS

The detailed description references the accompanying figures. The use of the same reference numbers in different instances in the description and the figures may indicate similar or identical items. In addition, the proportion and the relative scale of the elements provided in the figures are intended to illustrate various embodiments of the present disclosure and are not to be used in a limiting sense.

FIG. 1 illustrates an example of an equine sensor.

FIG. 2 is an isometric view of an equine sensor module.

FIG. 3 is a block hardware diagram of an equine sensor module.

FIG. 4 is a block hardware diagram of a computing device.

FIG. 5 is a block hardware diagram of a positioning device.

FIG. 6 is an isometric view of a positioning device.

FIG. 7 is a block hardware diagram of an equine sensor system.

FIG. 8 is a block hardware diagram of an equine sensor system.

FIG. 9 illustrates an example of a number of detection loops positioned along a racetrack.

FIG. 10 illustrates an example of a graph of an accelerometer magnitude over time.

FIG. 11 illustrates an example of a graph of acceleration in a lateral direction, a longitudinal direction, and a dorsal-ventral direction over time.

FIG. 12 illustrates an example of a graph of a minimum lateral acceleration, a maximum lateral acceleration, and an asymmetry index over time.

FIG. 13 illustrates an example of a flow diagram for storing and downloading equine data.

FIG. 14 illustrates a table of example equine data stored in a file.

FIG. 15 illustrates an example of a flow diagram for using an equine sensor and storing equine data.

### DETAILED DESCRIPTION

The present disclosure includes an equine sensor system comprising an equine sensor and a computing device. The equine sensor can include a strap removably coupled to a tail of an equine, an accelerometer configured to collect accelerometer data, and a transmitter coupled to the accelerometer configured to transmit the accelerometer data. The computing device can include a memory, a receiver configured to receive the accelerometer data, and a processor coupled to the memory and the receiver, wherein the processor is configured to determine a stride length of the equine based on the accelerometer data and positional data. An equine can be a horse, pony, donkey, mule, or zebra, for example.

Conventional animal monitors use sensors attached to equipment fastened to the animal (e.g., tack), which often leads to inaccurate measurements. For example, sensors can be coupled to a girth, which secures a saddle to an equine. When coupled to a girth, the sensors can have discontinuities in the recorded data due to interference between the sensors and the skin from the fur of the animal or vibration from the girth moving in response to the equine moving.

The equine sensor disclosed herein is configured to provide accurate and complete accelerometer data while easily and comfortably being worn by an equine. Although an equine sensor for an equine will be used as an example throughout this application, the equine sensor can be used on any tailed mammal and the equine sensor can measure and record various metrics including but not limited to acceleration. For example, the equine sensor can record a wide variety of metrics regarding the equine and its movement.

In various embodiments, the strap of the equine sensor has a cavity encased in plastic to receive the equine sensor. The strap can further include an attachment portion, for example a hook and loop fastener (e.g., Velcro), to couple the equine sensor to the tail of the equine in a manner that allows the equine sensor to be tightly held against the underside of the equine's tail.

The underside of an equine's tail is bare (e.g., hairless), which allows direct and repeatable contact with the skin without requiring that the equine be shaved. Direct and repeatable contact with the skin reduces discontinuities in the data. The tail of an equine can remain relatively stationary compared to other body parts of the equine when the equine is sleeping, eating, grazing, walking, trotting, and/or cantering, for example. Fastening the equine sensor to a more stationary part of the equine can minimize vibration, which can allow the equine sensor to record data more accurately. And, in cases where the tail is moved rapidly (e.g. during fly swatting for example), the mechanical signature of the tail movement is distinctive and uncorrelated with desired measurements and thereby easily filtered or otherwise removed from the recorded data.

The equine sensor can be included in an equine sensor system. The equine sensor system disclosed herein is configured to provide accurate and complete positional data. Positional data can be collected along with the accelerometer data by the equine sensor system to determine a stride length of an equine. For example, a positioning device of the equine sensor system can collect positional data via a global navigation satellite system (GNSS) receiver and transmit the positional data via a positioning device transmitter. A computing device of the equine sensor system can receive the accelerometer data and the positional data via a receiver and determine the stride length of the equine based on the accelerometer data and the positional data.

In a number of embodiments, the equine sensor system can include a transponder and a detection loop. The transponder can be coupled to a rider, the equine, or the strap. The transponder can transmit a unique signal that can be received at the detection loop, which can be an antenna positioned adjacent to a racetrack. The detection loop can receive and transmit the unique signal when the transponder passes the detection loop. The unique signal along with the accelerometer data can be used to determine the stride length of the equine.

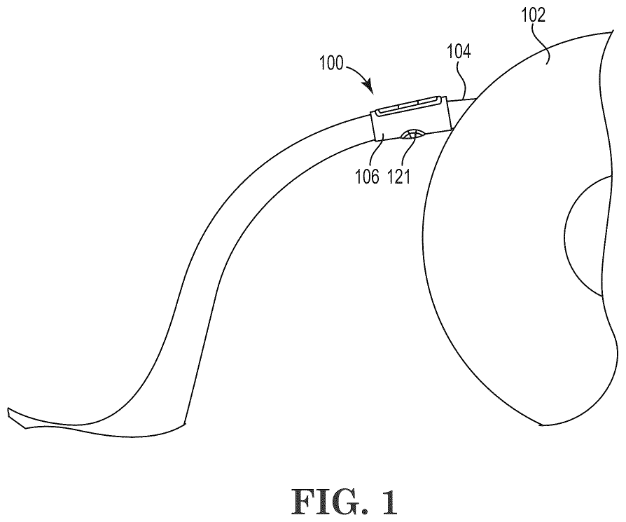

FIG. 1 illustrates an example of an equine 102 wearing an equine sensor 100. The equine sensor 100 is operable to record biometric and fitness information of an equine 102. The equine sensor 100 may be configured in a variety of ways. For instance, equine sensor 100 can be configured for use during equine competitions (e.g., racing, eventing, hunter jumper, dressage, barrel racing, trick riding, and/or rodeos), grazing, training, sleeping, during transport and trailering, resting, and/or trail riding.

As illustrated in FIG. 1, the equine sensor 100 includes a strap 106, which is shown in a looped (e.g., closed) configuration, wrapped around a tail 104 of the equine 102. The equine sensor 100 is removably coupled to a tail 104 of the equine 102 by the strap 106 circling the tail 104 of the equine 102.

Movement of the equine sensor 100 and/or the body of the equine 102 can cause discontinuities in data including accelerometer data and/or positional data recorded by an equine sensor module 121. The barrel of the equine 102, which is where many current equine sensors are attached, can move with the inhaling and exhaling of the equine 102. Equine sensors at the barrel of the equine 102, often are attached to the equine 102 via a girth, which can attach to a saddle of the equine 102. The weight of the saddle and/or rider can cause the girth and the equine sensor to vibrate and/or move, which can cause discontinuities in the data. A user may notice that the data is incorrect and try to tighten the girth in an attempt to get more accurate readings. Over cinching the girth may cause pain or discomfort to the equine 102 which could result in temporary or permanent physical or mental harm to the equine 102 and/or cause the equine 102 to react in a manner, which could be dangerous to a person.

As a common practice, girth located systems often are installed and removed with the saddle or other tack elements, and therefore have limitations as to when they provide data regarding equine health/wellness. Tail systems can be left on extended amounts of time independent of tacking and de-tacking processes. This allow better fitting into a standard training or competition workflow.

The tail 104 of the equine 102 can remain relatively stationary compared to other body parts, for example, the barrel of the equine 102. Accordingly, attaching the equine sensor 100 to the tail 104 of the equine 102 can reduce exposure of the equine sensor 100 to movement and/or vibration. As such, the equine sensor 100 attached to the tail 104 of the equine 102 can more accurately record data than current equine sensors.

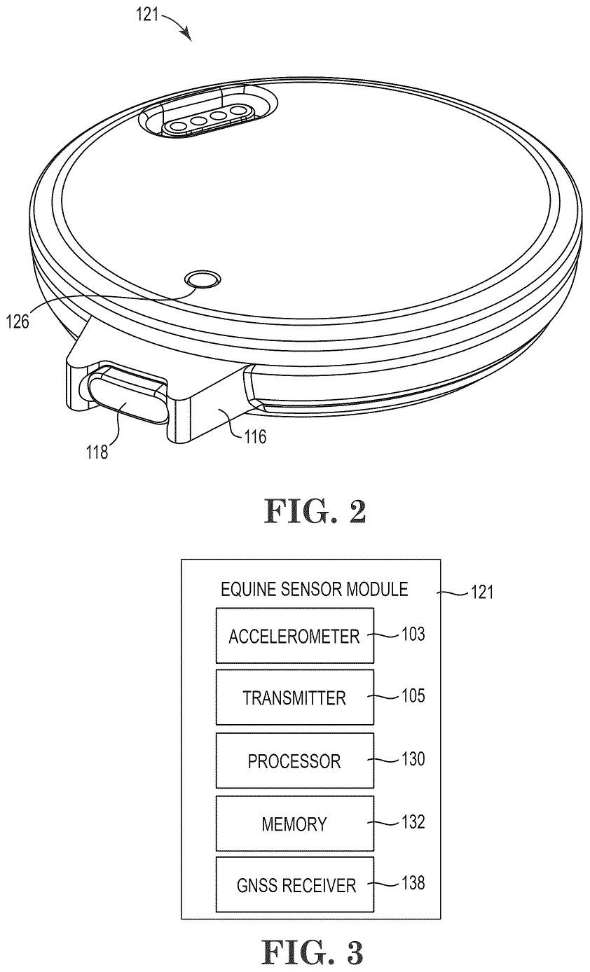

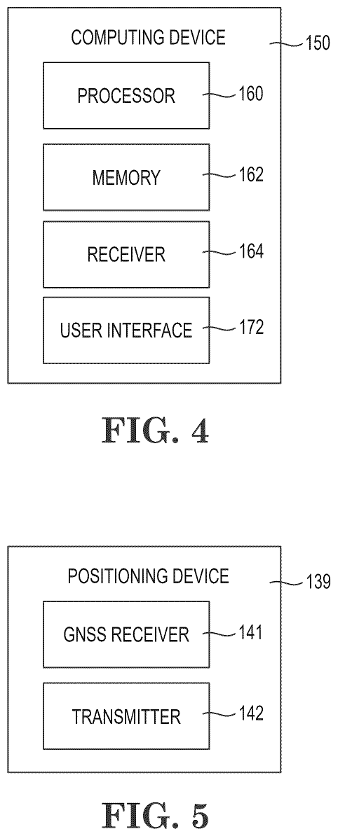

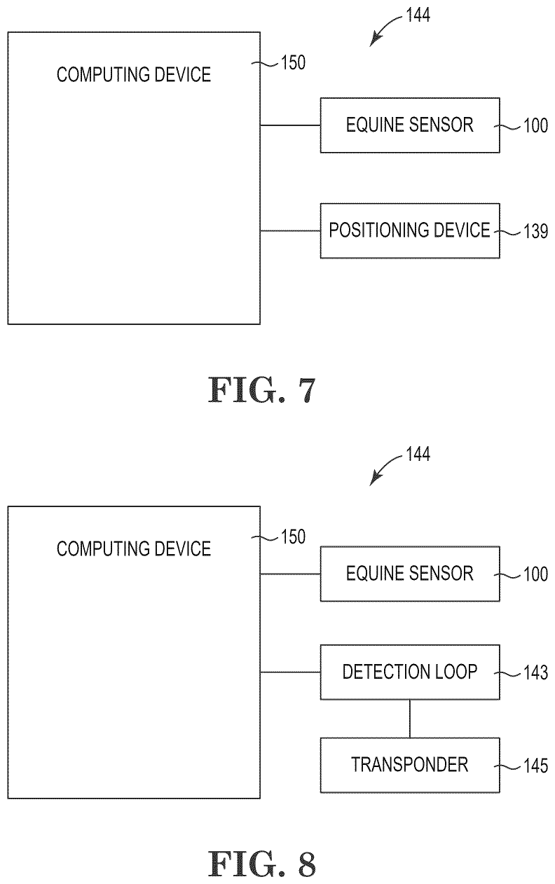

FIG. 2 is an isometric view of the equine sensor module 121. The equine sensor module 121 can include a tab 116, a button 118, and/or an LED 126. The tab 116 can consistently orient the equine sensor module 121 within a strap (e.g., strap 106 of FIG. 1). For example, equine sensor module 121 can be positioned such that the tab 116 can extend outward from the body of the equine sensor module 121 towards an end of a tail (e.g., tail 104 of FIG. 1) of an equine (e.g., equine 102 of FIGS. 1 and/or 9). This allows data from the equine sensor module 121 to be correlated with a direction of movement of the equine by a computing device (e.g., computing device 150 of FIGS. 4, 7, and/or 8) so that the direction of movement of the equine sensor module 121 is known.

The tab 116 can also act as a button guard to prevent the button 118 from being accidentally pressed while installed. For example, the tab 116 can include a cavity that houses the button 118, which covers a portion of the button 118 to prevent the button 118 from being pressed while the equine sensor module 121 is attached to an equine.

The button 118 can power on (e.g., turn on) and power off (e.g., turn off) the equine sensor module 121. A user can know when the equine sensor module 121 is powered on or powered off based on the LED 126. For example, the LED 126 can emit a light to indicate that the equine sensor module 121 is powered on or the LED 126 can emit a particular color light or flashing pattern to indicate that the equine sensor module 121 is powered on. In some embodiments, the LED 126 can indicate an issue, for example, a low battery, an error with the equine sensor module 121, and/or a connectivity issue between the equine sensor module 121 and the computing device by emitting a particular color light or flashing pattern.

FIG. 3 is a block hardware diagram of an equine sensor module 121. The equine sensor module 121 can include an accelerometer 103, a transmitter 105, a processor 130, a memory 132, and/or a GNSS receiver 138.

The processor 130 provides processing functionality for the equine sensor module 121 and can include any number of processors, micro-controllers, circuitry, such as an application specific integrated circuit (ASIC), field programmable gate array (FPGA) or other processing systems, and resident or external memory for storing data, executable code, and other information. In some embodiments, the processor 130 can execute one or more software programs embodied in a non-transitory computer readable medium (e.g., memory 132) that implement techniques described herein including collecting accelerometer data, transmitting the accelerometer data, collecting positional data, transmitting the positional data, and/or determining a stride length of an equine (e.g., equine 102 of FIGS. 1 and/or 9) based on the accelerometer data and the positional data. In some embodiments, the processor 130 can include circuitry, such as an ASIC, such that the functionality described herein can be provided by the processor 130 without the execution of separate software. The processor 130 is not limited by the materials from which it is formed or the processing mechanisms employed therein and, as such, can be implemented via semiconductor(s) and/or transistors (e.g., using electronic integrated circuit (IC) components), and so forth.

The memory 132 can be a tangible, computer-readable storage medium that provides storage functionality to store various data and/or program code associated with an operation, such as software programs and/or code segments, or other data to instruct the processor 130, and possibly other components of the equine sensor module 121, to perform the functionality described herein. The memory 132 can store data, such as program instructions for operating the equine sensor module 121 including its components, and so forth. The memory 132 can also store accelerometer data, positional data, stride length, stride frequency, stride and stance phases, distance traveled, stride consistency metric, and the like.

It should be noted that while a single memory 132 is described, a wide variety of types and combinations of memory (e.g., tangible, non-transitory memory) can be employed. The memory 132 can be integral with the processor 130, can comprise stand-alone memory, or can be a combination of both. Some examples of the memory 132 can include removable and non-removable memory components, such as random-access memory (RAM), read-only memory (ROM), flash memory (e.g., a secure digital (SD) memory card, a mini-SD memory card, and/or a micro-SD memory card), magnetic memory, optical memory, universal serial bus (USB) memory devices, hard disk memory, external memory, and so forth. In a number of embodiments, the equine sensor module 121 and/or the memory 132 can include removable integrated circuit card (ICC) memory, such as memory provided by a subscriber identity module (SIM) card, a universal subscriber identity module (USIM) card, a universal integrated circuit card (UICC), and so on.

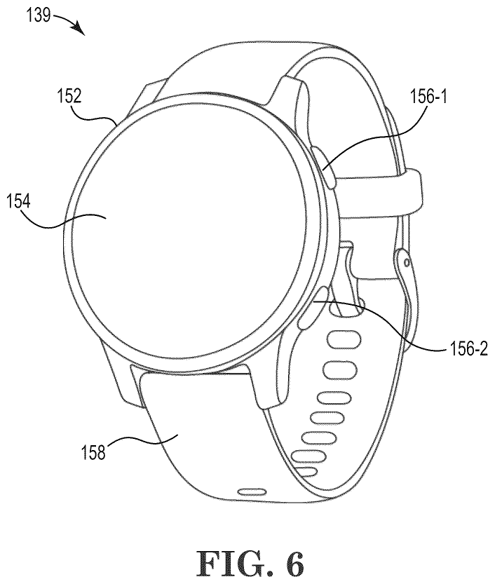

The transmitter 105 can receive control signals and/or other communications from and/or transmit control signals and/or other communication to, for example, a computing device (e.g., computing device 150 of FIGS. 4, 7, and/or 8), positioning device (e.g., positioning device 139 of FIGS. 5, 6, and/or 7), and/or detection loop (e.g., detection loop 143 of FIG. 8). The transmitter 105 can be communicatively coupled to the computing device, the positioning device, and/or the detection loop via a wired or wireless connection. Examples of wireless transmission include electromagnetic and optical, among others. Accordingly, the transmitter 105 can be a wireless transmitter configured to transmit and/or receive data, including accelerometer data, positional data, stride length, stride frequency, stride and stance phases, distance traveled, and/or stride consistency metric via Bluetooth and/or a cellular network, for example.

The equine sensor module 121 can transmit data via the transmitter 105 to a rider's local device for instantaneous reception, to a trainer device located in proximity to the equine 102, or to a cloud-based storage and processing system for near real-time access from any internet-connected computing device. In some embodiments, transmission latency can be adjustable such that parameters associated with higher time sensitivity are prioritized and transmitted at increased frequency relative to less time-sensitive parameters. The processor 130 can manage this prioritization dynamically to optimize communication performance based on data type and transmission conditions.

The accelerometer 103 can be any inertial sensor, including a gyroscope, that can detect and collect an orientation, change in orientation, direction, change in direction, position, and/or change in position of the equine sensor module 121, referred to herein as accelerometer data. Acceleration magnitudes in a dorsal-ventral direction (e.g., back-to-belly), lateral direction (e.g., shoulder-to-shoulder), and/or a longitudinal direction (e.g., head-to-tail) of the equine can be included in the accelerometer data collected by the accelerometer 103. It will be appreciated by those of ordinary skill in the art that a three-dimensional vector describing a movement of the equine sensor module 121 through three-dimensional space can be established by combining the acceleration magnitudes in the dorsal-ventral direction, the lateral direction, and/or the longitudinal direction using known methods. Single and multiple axis models of the accelerometer 103 are capable of detecting magnitude and direction of acceleration as a vector quantity and may be used to sense orientation and/or coordinate acceleration of the equine.

The equine sensor module 121 can also include a GNSS receiver 138 (e.g., a global positioning system (GPS) receiver, assisted-GPS, software defined (e.g., multi-protocol) receiver, or the like) or any location or position determining component that is configured to collect positional data for the equine sensor module 121 (e.g., geographic coordinates of at least one reference point on the equine sensor module 121). The GNSS receiver 138 generally determines a current geolocation of the equine sensor module 121 and may process a first electronic signal, such as radio frequency (RF) electronic signals, from a GNSS such as GPS primarily used in the United States, the GLONASS system primarily used in the Soviet Union, or the Galileo system primarily used in Europe. The GNSS receiver 138 may include satellite navigation receivers, processors, controllers, other computing devices, or combinations thereof, and memory. The GNSS receiver 138 may be in electronic communication with an antenna (not shown) that may wirelessly receive an electronic signal from one or more of the previously-mentioned satellite systems and provide the electronic signal to the GNSS receiver 138. The GNSS receiver 138 may process the electronic signal, which includes data and information, from which geographic information such as the current geolocation is determined. The current geolocation may include geographic coordinates, such as the latitude and longitude, of the current geographic location of the equine sensor module 121. The GNSS receiver 138 may communicate the current geolocation to the processor 130. Generally, the GNSS receiver 138 is capable of determining continuous position, velocity, time, and direction (heading) information.

In various embodiments, the GNSS receiver 138 can be located within the equine sensor module 121, positioned elsewhere on the equine 102, or incorporated into a wearable or separate device carried by the rider. The GNSS receiver 138 can also interface with stationary positioning components arranged along a course to supplement the mobile positioning data and increase overall accuracy. In some examples, the GNSS receiver 138 can communicate with a correction source such as a real-time kinematic (RTK) system to refine positional precision.

In certain embodiments, the sensor 100 can communicate with external track timing systems, such as those provided by MYLAPS®, to enhance timing and positional accuracy. The equine sensor module 121 can transmit or receive timing, sensor, and positioning information to and from the timing system to correct recorded data with precise timing events recorded at the track. In some configurations, the track timing system can receive position information directly from the equine sensor 100 to supplement its own timing and location data, thereby generating enhanced positional and performance information for the equine 102 and other participants.

Although not illustrated in FIG. 3, the equine sensor module 121 can include a number of other sensors. For example, the equine sensor module 121 can further include a heart rate sensor that can generate a pulse oximeter (spO2) signal and/or determine a respiration rate, a thermometer, and/or a barometric sensor, among other sensors.

FIG. 4 is a block hardware diagram of a computing device 150.

The computing device 150 can include a processor 160, a memory 162, a receiver 164, and/or a user interface 172. The computing device 150 can be, but is not limited to, a smartphone, a tablet, a laptop, a desktop computer, or a cloud computing device.

The processor 160 provides processing functionality for the computing device 150 and can include any number of processors, micro-controllers, circuitry, such as an application specific integrated circuit (ASIC), FPGA or other processing systems, and resident or external memory for storing data, executable code, and other information. In some embodiments, the processor 160 can execute one or more software programs embodied in a non-transitory computer readable medium (e.g., memory 162) that implement techniques described herein including receiving accelerometer data and/or positional data via receiver 164 and/or determining a stride length of an equine (e.g., equine 102 of FIGS. 1 and/or 9) based on the accelerometer data and the positional data. In some embodiments, the processor 160 can include circuitry, such as an ASIC, such that the functionality described herein can be provided by the processor 160 without the execution of separate software.

In a number of embodiments, the processor 160 can determine a stride frequency of the equine based on acceleration magnitudes in a dorsal-ventral direction of the equine from the accelerometer data. The stride frequency of the equine can be used by the processor 160 to characterize stride and stance phases of the equine. The processor 160 can determine a distance traveled by the equine based on the positional data and determine the stride length of the equine by dividing the distance traveled by the equine by a number of strides taken by the equine based on the stride frequency of the equine. Further, a stride consistency metric of the equine can be determined by the processor 160 based on a number of stride lengths of the equine throughout a work session.

The processor 160 is not limited by the materials from which it is formed or the processing mechanisms employed therein and, as such, can be implemented via semiconductor(s) and/or transistors, and so forth. The one or more processors 160 may be adapted and configured to execute any of a number of software applications and/or any of a number of software routines residing in memory 162, in addition to other software applications. One of the number of applications may be a client application that may be implemented as a series of machine-readable instructions for performing the various functions associated with implementing the performance of an equine sensor system (e.g., equine sensor system 144 of FIGS. 7 and/or 8) as well as receiving information at, displaying information on, and transmitting information from the computing device 150. The client application may function to implement a system wherein the front-end components communicate and cooperate with back-end components. The client application may include machine-readable instructions for implementing a user interface 172 to allow a user to input commands to, and receive information from, the computing device 150. One of the plurality of applications may be a native web browser, such as Apple's Safari®, Google Android™ mobile web browser, Microsoft Edge® for Mobile, Opera Mobile™, that may be implemented as a series of machine-readable instructions for receiving, interpreting, and displaying web page information from a server device or other back-end components while also receiving inputs from the computing device 150. Another application of the plurality of applications may include an embedded web browser that may be implemented as a series of machine-readable instructions for receiving, interpreting, and displaying web page information from the server device or other back-end components within the client application.

In certain embodiments, data collected by the equine sensor module 121 can be transmitted for integration into live or recorded video content. The transmitted data can be overlaid or synchronized with closed-circuit video feeds, broadcast television signals, or other video production systems associated with an equine event. The processor 130 and/or the computing device 150 can format and transmit telemetry data corresponding to the equine 102 and/or rider in real time for display within video productions designed for audience viewing. In some configurations, such telemetry data can include acceleration, stride frequency, stride length, positional data, and performance metrics, which can be combined with other data sources in a broadcast control system to provide synchronized horse and jockey telemetry during equine competitions, training sessions, or exhibitions.

The client applications or routines may include an accelerometer routine that determines the acceleration and direction of movements of an equine sensor module (e.g., equine sensor module 121 of FIGS. 1, 2, and/or 3), which correlate to the acceleration, direction, and movement of the equine. The accelerometer routine may receive and process data from an accelerometer (e.g., accelerometer 103 of FIG. 3) to determine one or more vectors describing the motion of the equine for use with the client application.

The client applications or routines may further include a positioning routine that coordinates with a GNSS receiver (GNSS receiver 138 of FIG. 3) of the equine sensor module, a GNSS receiver (GNSS receiver 141 of FIG. 5) of a positioning device (e.g., positioning device of FIGS. 5, 6, and/or 7), and/or a detection loop (e.g., detection loop 143 of FIG. 8) to determine or obtain positional data for use with one or more of the plurality of applications, such as the client application, or for use with other routines.

The user may also launch or initiate any other suitable user interface application to access a server device to implement an equine monitoring process. Additionally, a user may launch the client application from the computing device 150 to access the server device to implement the equine monitoring process.

In a number of embodiments, the computing device 150 and/or a server may perform one or more processing functions remotely that may otherwise be performed by the equine sensor module, the positioning device, and/or the detection loop. In such embodiments, the computing device 150 or server may include a number of software applications capable of receiving equine data gathered from the equine senor module, the positioning device, and/or the detection loop including, but not limited to, accelerometer data and positional data. For example, the equine sensor module, the positioning device, and/or the detection loop may gather equine data from sensors as described herein, but instead of using the equine data locally, the equine sensor module, the positioning device, and/or the detection loop may send the equine data to the computing device 150 or the server for remote processing.

The computing device 150 or the server may perform the analysis of the gathered equine data to determine a stride length of the equine, for example. The accelerometer data and the positional data may be sent to the server device and include a request for analysis, where the stride length determined by the server device is returned to computing device 150, the positioning device, and/or the equine sensor module.

The memory 162 can be a tangible, computer-readable storage medium that provides storage functionality to store various data and/or program code associated with an operation, such as software programs and/or code segments, or other data to instruct the processor 160, and possibly other components of the computing device 150, to perform the functionality described herein. The memory 162 can store data, such as program instructions for operating the computing device 150 including its components, and so forth. The memory 162 can also store accelerometer data, positional data, stride length, stride frequency, stride and stance phases, distance traveled, stride consistency metrics, and the like. In a number of embodiments, the memory 162 can store an application, which enables a user to view and analyze equine data received from the equine sensor module, the positioning device, the detection loop, and/or the computing device 150.

It should be noted that while a single memory 162 is described, a wide variety of types and combinations of memory can be employed. The memory 162 can be integral with the processor 160, can comprise stand-alone memory, or can be a combination of both. Some examples of the memory 162 can include removable and non-removable memory components, such as RAM, ROM, flash memory, magnetic memory, optical memory, USB memory devices, hard disk memory, external memory, and so forth. In a number of embodiments, the computing device 150 and/or the memory 162 can be ICC memory, such as memory provided by a SIM card, a USIM card, a UICC, and so on.

A communication module illustrated as receiver 164 in FIG. 4 may enable computing device 150 to communicate with the equine sensor module, the positioning device, the detection loop, and/or the server device via any suitable wired or wireless communication protocol independently or using I/O circuitry. The wired or wireless network may include a wireless telephony network (e.g., Global System for Mobile communications (GSM), Code-Division Multiple Access (CDMA), Long-Term Evolution (LTE), etc.), one or more standard of the Institute of Electrical and Electronics Engineers (IEEE), such as 802.11 or 802.16 (Wi-Max) standards, Wi-Fi standards promulgated by the Wi-Fi Alliance, Bluetooth standards promulgated by the Bluetooth Special Interest Group, a near field communication standard (e.g., ISO/IEC 18092, standards provided by the NFC Forum, etc.), satellite communication networks like Starlink, 5G NTN, Iridium, and so on. Wired communications are also contemplated such as through universal serial bus (USB), Ethernet, serial connections, and so forth.

The computing device 150 may be configured to communicate via one or more networks with a cellular provider and an Internet provider to receive mobile phone service and various content, respectively. Content can comprise map data, which may include route information, web pages, services, music, photographs, video, email service, instant messaging, device drivers, real-time and/or historical weather data, instruction updates, and so forth.

The receiver 164 can receive control signals and/or other communications from, for example, the equine sensor module, the positioning device, the detection loop, and/or the server. The receiver 164 can be communicatively coupled to the equine sensor module, the positioning device, the detection loop, and/or the server via a wired or wireless connection. Accordingly, the receiver 164 can be a wireless receiver configured to receive data, including accelerometer data from the equine sensor module and/or positional data from the positioning device and/or the detection loop via Bluetooth and/or a cellular network, for example.

A user may interact with the displayed data via user interface 172, which may include a “soft” keyboard that is presented on a display of the computing device 150, an external hardware keyboard communicating via a wired or a wireless connection (e.g., a Bluetooth keyboard), and/or an external mouse, or any other suitable user-input device or component. The user interface 172 may include or communicate with a microphone capable of receiving voice input from a user as well as the display having a touch input.

FIG. 5 is a block hardware diagram of a positioning device 139. The positioning device 139 can be, but is not limited to, a smartwatch, a wearable device, a smartphone, or the like. The positioning device 139 can include a GNSS receiver 141 and/or a transmitter 142.

The positioning device 139 can include the GNSS receiver 141 (e.g., a GPS receiver, assisted-GPS, software defined receiver, or the like) or any location or position determining component that is configured to collect positional data for the positioning device 139 (e.g., geographic coordinates of at least one reference point on the positioning device 139). The GNSS receiver 141 generally determines a current geolocation of the positioning device 139 and may process a first electronic signal, such as radio frequency (RF) electronic signals, from a GNSS such as GPS primarily used in the United States, the GLONASS system primarily used in the Soviet Union, or the Galileo system primarily used in Europe. The location GNSS receiver 141 may include satellite navigation receivers, processors, controllers, other computing devices, or combinations thereof, and memory. The GNSS receiver 141 may be in electronic communication with an antenna (not shown) that may wirelessly receive an electronic signal from one or more of the previously-mentioned satellite systems and provide the electronic signal to the GNSS receiver 141. The GNSS receiver 141 may process the electronic signal, which includes data and information, from which geographic information such as positional data including the current geolocation is determined. The positional data may include geographic coordinates, such as the latitude and longitude, of the current geographic location of the positioning device 139. The GNSS receiver 141 may communicate the positional data to a computing device (e.g., computing device 150 of FIGS. 4, 7, and/or 8) and/or an equine sensor module (e.g., equine sensor module 121 of FIGS. 1, 2, and/or 3). Generally, the GNSS receiver 141 is capable of determining continuous position, velocity, time, and direction (heading) information.

The transmitter 142 can receive control signals and/or other communications from and/or transmit control signals and/or other communication to, for example, the computing device and/or the equine sensor module. The transmitter 142 can be communicatively coupled to the computing device and/or the equine sensor module via a wired or wireless connection. Accordingly, the transmitter 142 can be a wireless transmitter configured to transmit data, including positional data via Bluetooth and/or a cellular network, for example.

FIG. 6 is an isometric view of a positioning device 139. The positioning device 139 can be paired with an equine sensor module (e.g., equine sensor module 121 of FIGS. 1, 2, and/or 3) and/or a computing device (e.g., computing device 150 of FIGS. 4, 7, and/or 8) to receive and/or transmit data. The positioning device 139 can transmit data, including positional data as previously described in connection with FIG. 5. In a number of embodiments, the positioning device 139 can store data in memory and/or convey data to a user via a user interface and/or a speaker. For example, the positioning device 139 can present information based on the positional data and/or accelerometer data of an equine (e.g., equine 102 of FIGS. 1 and/or 9).

When the positioning device 139 is a smartwatch, as illustrated in FIG. 6, the positioning device 139 can include a band 158 coupled to a housing 152 including a number of buttons 156-1, 156-2 and a display 154. The buttons 156-1, 156-2 are configured to control a number of functions of the positioning device 139.

The band 158 may be removably secured to the housing 152 via attachment of securing elements to corresponding connecting elements. Examples of securing elements and/or connecting elements include, but are not limited to hooks, latches, clamps, snaps, and the like. The band 158 may be made of a lightweight and resilient thermoplastic elastomer and/or a fabric, for example, such that the band 158 may encircle a portion of a user without discomfort while securing the housing 152 to the user. The band 158 may be configured to attach to various portions of a user, such as a user's leg, waist, wrist, forearm, and/or upper arm.

The display 154 may include a liquid crystal display (LCD), a thin film transistor (TFT), a light-emitting diode (LED), an active-matrix organic light-emitting diode (AMOLED), a light-emitting polymer (LEP), and/or a polymer light-emitting diode (PLED). However, embodiments are not so limited. The display 154 may be capable of displaying text and/or graphical information. The display 154 may be provided with a touch screen to receive input (e.g., data, commands, etc.) from a user. For example, a user may operate the positioning device 139 by touching the touch screen and/or by performing gestures on the display 154. In some embodiments, the display 154 may be a capacitive touch screen, a resistive touch screen, an infrared touch screen, or any combinations thereof.

FIG. 7 is a block hardware diagram of an equine sensor system 144. The equine sensor system 144 can include a computing device 150, an equine sensor 100, and/or a positioning device 139. The computing device 150, the equine sensor 100 including an equine sensor module (e.g., equine sensor module 121 of FIGS. 1, 2, and/or 3) and the positioning device 139 can be communicatively coupled via a network. The computing device 150 can receive accelerometer data from the equine sensor 100 and positional data from the positioning device 139. As previously discussed, a stride length can be determined by the computing device 150 based on the accelerometer data and the positional data.

In a number of embodiments, the positioning device 139 can be attached to a rider of an equine (e.g., equine 102 of FIGS. 1 and/or 9) while the equine sensor 100 is attached to the equine. For example, the positioning device 139 can be removably coupled to clothing or equipment of the rider while the equine sensor 100 is removably coupled to a tail (e.g., tail 104 of FIG. 1) of the equine. However, in some examples, the positioning device 139 can be coupled or removably coupled to the equine or tack of the equine and/or the positioning device 139 can be included in the equine sensor 100.

The computing device 150 can be included in the equine sensor 100, the positioning device 139, or external to the equine sensor 100 and the positioning device 139. For example, the computing device 150 can be located and used by a third-party observer including a trainer, a spectator, a veterinarian, an announcer, and/or a judge to determine a stride length and/or display the stride length, among other data about the equine.

In some examples, the equine sensor system 144, although not shown, can include a number of other sensors. For example, a jockey sensor can be coupled to a jockey or the equipment of the jockey and communicatively coupled to the computing device 150. The jockey sensor can be coupled to or included in a whip of the jockey to determine a number of times the whip was used, where on the equine the whip made contact, when the whip was used, and/or a location along the racetrack where the whip was used. The data from the jockey sensor in the whip can be linked to performance metrics of the equine by the computing device 150 to determine when and/or if the whip increased or decreased performance of the equine. It can also be used to ensure rules regarding the whip are not violated. For example, in the United States, a whip cannot be used more than six times during a race, can only be used on the hindquarters of the equine, can only be used in an underhanded or backhanded motion, and the equine must be given time to respond between strikes from the whip.

In a number of embodiments, the jockey sensor can be in or coupled to the boots of the jockey or coupled to the stirrup irons, stirrup leathers, or saddle. The jockey sensor can detect the positioning of the jockey to determine stances (e.g., seats) of the jockey throughout the race. The data from the jockey sensor coupled to the boots, stirrup irons, stirrup leathers, or saddle can be linked to performance metrics of the equine by the computing device 150 to determine when and/or if each stance increased or decreased performance of the equine.

FIG. 8 is a block hardware diagram of an equine sensor system 144. The equine sensor system 144 can include a computing device 150 communicatively coupled to an equine sensor 100 and/or a detection loop 143. The detection loop 143 can be communicatively coupled to a transponder 145.

The transponder 145 can be removably coupled to a rider of an equine (e.g., equine 102 of FIGS. 1 and/or 9), the equine, or a strap (e.g., strap 106 of FIG. 1) of an equine sensor (e.g., equine sensor 100 of FIG. 1). The transponder 145 can transmit a unique signal.

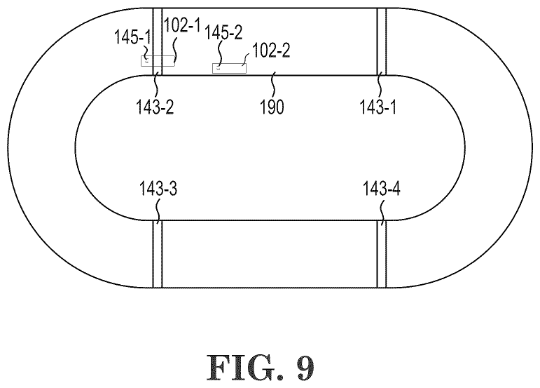

The detection loop 143 can receive and transmit the unique signal to a decoder to identify which transponder 145 passed and at what time in response to the transponder 145 passing the detection loop 143. In some examples, the decoder can be included in the computing device 150, communicatively coupled to the computing device 150, or the functionality of the decoder can be provided by the computing device 150 although not a discrete component thereof. The detection loop 143 can be setup adjacent to a racetrack (e.g., racetrack 190 of FIG. 9) so that the detection loop 143 receives the unique signal when the transponder 145 attached to the rider, the equine, or strap passes or goes through the detection loop 143. In some examples, the detection loop 143 can be an antenna. The antenna can be coupled to a loop of wire embedded in the racetrack or ground that generates a low-frequency alternating electromagnetic field. When the transponder 145 passes through or above the detection loop 143, the alternating current in the detection loop 143 creates an electromagnetic field. A coil of the transponder 145 picks up energy from the electromagnetic field, which induces a current that powers the transponder 145. When powered, the transponder 145 can transmit the unique signal, which can include a precise timestamp based on an internal clock of the transponder 145 to the detection loop 143.

The computing device 150, as previously discussed in connection with FIG. 4, can include a processor (e.g., processor 160 of FIG. 4), a memory (e.g., memory 162 of FIG. 4), and a receiver (e.g., receiver 164 of FIG. 4). The receiver can receive accelerometer data and/or the unique signal. The processor can determine a stride length of the equine based on the accelerometer data and the unique signal. The processor can determine a time the transponder 145 passes the detection loop 143 based on the unique signal and determine positional data based on the time the transponder 145 passes the detection loop 143. The positional data and the accelerometer can then be used by the processor to determine the stride length of the equine, among other information about the equine.

FIG. 9 illustrates an example of a number of detection loops 143-1, 143-2, 143-3, 143-4 positioned along a racetrack 190. FIG. 9 further includes a number of equines 102-1, 102-2. Transponder 145-1 is coupled to equine 102-1 or the rider of equine 102-1 while transponder 145-2 is coupled to equine 102-2 or the rider of equine 102-2.

Transponder 145-1, as illustrated in FIG. 9, has passed detection loop 143-2, accordingly detection loop 143-2 has received and transmitted a unique signal from transponder 145-1. An additional detection loop, for example, detection loop 143-3 can receive and transmit the unique signal from transponder 145-1 in response to the transponder 145-1 passing the additional detection loop 143-3.

In a number of embodiments, an additional transponder, for example, transponder 145-2 can transmit a different unique signal that is different from the unique signal of transponder 145-1. When transponder 145-2 passes detection loop 143-2, detection loop 143-2 will receive and transmit the different unique signal from transponder 145-2.

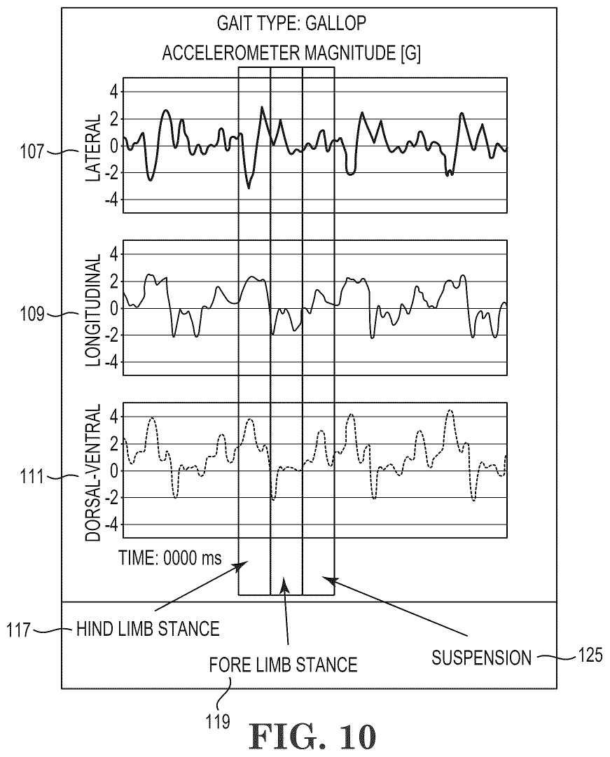

FIG. 10 illustrates an example of a graph of an accelerometer magnitude over time. The accelerometer data used to create the graph of the accelerometer magnitude over time can be from an accelerometer (e.g., accelerometer 103 of FIG. 3) of an equine sensor module (e.g., equine sensor module 121 of FIGS. 1, 2, and/or 3). The accelerometer data along with positional data collected by a GNSS receiver (e.g., GNSS receiver 138 of FIG. 3, GNSS receiver 141 of FIG. 5) or a detection loop (e.g., detection loop 143 of FIGS. 8 and/or 9) can be utilized to detect and calculate stride frequency, stride length, and lateral asymmetry, establishing a unique profile for each equine (e.g., equine 102 of FIGS. 1 and/or 9) across multiple workouts.

Lateral asymmetry may be a measurement of lateral lean, potentially indicating limb compromise or hind-end lameness. Stride length, derived from positional data and stride frequency data, can be used, for example, to signal front-limb issues (e.g., if the stride length determined from particular data for a particular equine is shortened versus the stride length determined from previous data for the particular equine). Each workout may be classified as “green,” “yellow,” or “red” by comparing these metrics to historical distributions and industry testing standards, or any other categorization technique, with deviations potentially prompting further evaluation. This method may allow for early detection of gait abnormalities and support proactive equine health management.

An equine sensor system (e.g., equine sensor system 144 of FIGS. 7 and/or 8) is also configured to calculate metrics related to movement and biomechanical performance, including gait type detection and speed. By analyzing the specific motion patterns and timing, the equine sensor system can distinguish between walking, trotting, cantering, and galloping, providing insight into stride frequency and speed for each gait. Additionally, the equine sensor system offers an asymmetry index (e.g., asymmetry index 135 of FIG. 12), allowing for the detection of imbalances in movement that may indicate early signs of injury or fatigue.

Advanced stride dynamics and injury early-warning metrics can enhance the equine sensor system's ability to monitor and alert for potential health issues. Through the analysis of stride dynamics, the equine sensor system assesses factors such as stride length, ground contact time, and impact force, which can reveal subtle deviations from normal patterns. An injury early-warning feature can operate in both real-time and post-activity modes, enabling immediate feedback during exercise and detailed analysis after sessions. This function supports early detection of injury risks, aiding in timely intervention and promoting optimal equine health management.

As described in more detail with respect to FIG. 12, the equine sensor system can include an asymmetry index metric, which may measure lateral force differences between the left and right rear limbs. This metric can be observed over time to detect subtle changes in gait that may be associated with potential injury or lameness. By providing data on gait symmetry, the asymmetry index may support assessments of movement quality and offer an early indication of deviations from typical patterns, which can aid in preventive health measures and may reduce the risk of undiagnosed lameness.

Additionally, the equine sensor system's advanced stride dynamics metric may provide detailed information on various gait characteristics across different speeds. For each stride, it can measure stride length, stance phase including separate assessments for hind and forelimb stance, and suspension phase. It may also record acceleration magnitudes in the lateral, longitudinal, and dorsal-ventral directions, creating a comprehensive profile of each gait. This level of detail may allow riders and trainers to assess movement efficiency and symmetry, as well as identify factors that could impact performance or potentially signal the onset of injury.

The gallop gait may be decomposed into distinct phases, each identifiable through specific acceleration patterns that the equine sensor system can capture. The hind limb stance phase 117 is marked by the right rear hoof making ground contact and ending as the left rear hoof lifts off. During the hind limb stance phase 117, a “scissor” motion occurs in the lateral direction 107 due to the equine pushing off each hind hoof. The hind limb stance phase 117 may also show positive acceleration in the longitudinal direction 109, indicating forward force applied through the rear hooves.

The fore limb stance phase 119 begins when the right front hoof touches down and ends as the left front hoof lifts off. This phase is characterized by two brief negative longitudinal direction 109 accelerations corresponding to the front hooves planting on the ground. Additionally, a negative dorsal-ventral direction 111 acceleration may occur as the equine rotates its hindquarters inward to prepare for the upcoming stride.

Finally, the suspension phase 125 begins when the left front hoof lifts off and ends as the right rear hoof touches down. In the suspension phase 125, all four hooves are off the ground, and a positive dorsal-ventral direction 111 acceleration may be captured as the equine briefly accelerates and then decelerates the forward motion of its hindquarters to prepare for the next hind limb stance phase 117. The dorsal-ventral direction 111 acceleration during this phase is measurable due to the equine sensor's position on the equine's tail, providing a clear view of the dynamics during the suspension phase 125.

The analysis of accelerometer data enables a detailed characterization of stride and stance phases for each equine during gallop, facilitating the calculation of stride frequency over any specified timeframe. Each observable movement in video footage corresponds to specific force transitions along one or more of the three measured axes including the lateral direction 107, the longitudinal direction 109, and the dorsal-ventral direction 111. This allows each phase of the gallop gait to be mapped with precision, providing insight into the equine's performance and movement dynamics. The specific force transitions may be represented in units of gravitational force (g-force), for example.

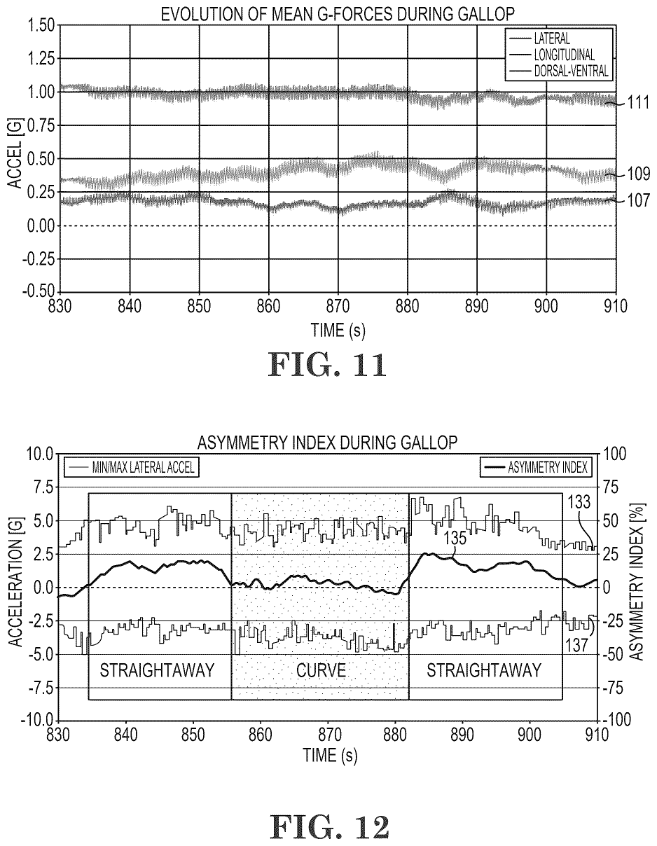

FIG. 11 illustrates an example of a graph of acceleration in a lateral direction 107, a longitudinal direction 109, and a dorsal-ventral direction 111 over time. During training sessions, breezes, or races, accelerometer data is collected as a measure of acceleration over time, which can be indicative of force over time. By applying a gait detection algorithm alongside positional data, a defined measurement window can be established to generate a gallop profile unique to the equine's performance at a particular racetrack. This gallop profile can be developed as a moving average (e.g., mean) for each axis component of the accelerometer, creating a consistent baseline that can be used to monitor changes or improvements in stride dynamics and overall gait efficiency.

FIG. 12 illustrates an example of a graph of a minimum lateral acceleration 137, a maximum lateral acceleration 133, and an asymmetry index 135 over time. The lateral direction (e.g., lateral direction 107 of FIGS. 10 and/or 11) and dorsal-ventral direction (e.g., dorsal-ventral direction 111 of FIGS. 10 and/or 11) of an accelerometer (e.g., accelerometer 103 of FIG. 3) are key for assessing asymmetry and weight-bearing forces during a gallop, providing metrics that may correlate directly with equine health indicators. The asymmetry index 135 is a primary metric used to establish the equine's unique profile and track potential health issues. The asymmetry index 135 measures the equine's lateral “lean,” with positive values indicating a leftward lean and negative values indicating a rightward lean. Such lean patterns can suggest, for example, a compromised limb on one side of the equine or the other. With an equine sensor (e.g., equine sensor 100 of FIGS. 1, 7, and/or 8) positioned at a tail (e.g., tail 104 of FIG. 1) of an equine (e.g., equine 102 of FIGS. 1 and/or 9), persistent deviations in the asymmetry index 135 are especially relevant for detecting hind-end lameness.

The asymmetry index 135 time series averaged over workout sessions provides a long-term distribution profile unique to each equine. Deviations from this baseline profile, quantified in standard deviations from the mean, are categorized as “green”, “yellow”, and “red” to indicate severity levels in health monitoring. For example, the “green” category can include indices within two standard deviations of the mean, indicating no current cause for concern, the “yellow” category can include indices beyond two standard deviations from the mean, indicating a significant deviation that may warrant further analysis, and the “red” category can include indices beyond three standard deviations, which signal an immediate need for veterinary evaluation and may necessitate halting the equine's training or competition. The red category reflects a near-certain link to a physical abnormality that requires prompt intervention.

Stride length serves as a secondary parameter for detecting gait abnormalities, with a decrease in stride length potentially indicating front-limb lameness or injury. Using dorsal-ventral accelerometer data, an equine sensor system (e.g., equine sensor system 144 of FIGS. 7 and/or 8) using, for example, a gait algorithm can detect stride frequency, identifying each complete cycle of movement through specific patterns in vertical acceleration. By pairing this stride frequency data with positional data, the equine sensor system can calculate the stride length in meters throughout a workout session, offering a metric for monitoring stride consistency.

This computed stride length, when analyzed over time, provides a clear measure of any deviations from an equine's typical gait pattern. As such, prolonged reductions in stride length could signify physical discomfort or injury in the front limbs. This integration of stride frequency with positional data creates a continuous, precise measure of stride length, supporting the early detection of gait irregularities.

Using data from a GNSS receiver (e.g., GNSS receiver 141 of FIG. 5) in a companion smartwatch, for example, the equine sensor system can calculate a total distance traveled during any given workout. This distance measurement, in conjunction with the recorded number of gallop strides, allows for the calculation of stride length across the session. By dividing the distance traveled by the number of strides taken, the equine sensor system provides an average stride length metric, offering insight into gait efficiency and potential abnormalities.

This calculation process enables consistent monitoring of stride length across different workouts, supporting the detection of trends or deviations from the equine's baseline performance. Persistent decreases in stride length, for example, may be indicative of emerging gait issues or discomfort, allowing for proactive management of the equine's health and performance.

To establish a reliable baseline, stride length data can be recorded over ten to thirty workouts, creating a distribution specific to each equine. Future workouts can then be measured against this historical distribution, identifying deviations from the mean. Based on the degree of deviation, each session may be classified into a “green,” “yellow,” or “red” status, similar to the system used for the asymmetry index 135.

Either a critical kinetic parameter, a stride length, or an asymmetry index 135 can trigger a “yellow” or “red” warning independently, flagging the workout for further evaluation. For instance, if a workout remains “green” on the asymmetry index 135 but shows a “red” deviation in stride length, the workout is still classified as “red” overall. This dual-parameter approach allows for early identification of potential gait abnormalities, enabling timely intervention and management.

The equine sensor system can connect to various devices, including a smartwatch worn by the rider, a smartphone application, a tablet, or a computer, to present real-time equine data including kinematic data and enable analysis after sessions. Metrics such as stride length, asymmetry index 135, and stride frequency are accessible on the smartwatch, allowing riders to monitor gait dynamics during sessions. Alerts for deviations, labeled as “yellow” or “red,” can prompt riders to adjust training intensity or take breaks. The smartwatch or any wearable device may also use vibrations or audio cues to provide these alerts, allowing riders to receive feedback without visually checking the device, thus staying focused on the equine's performance.

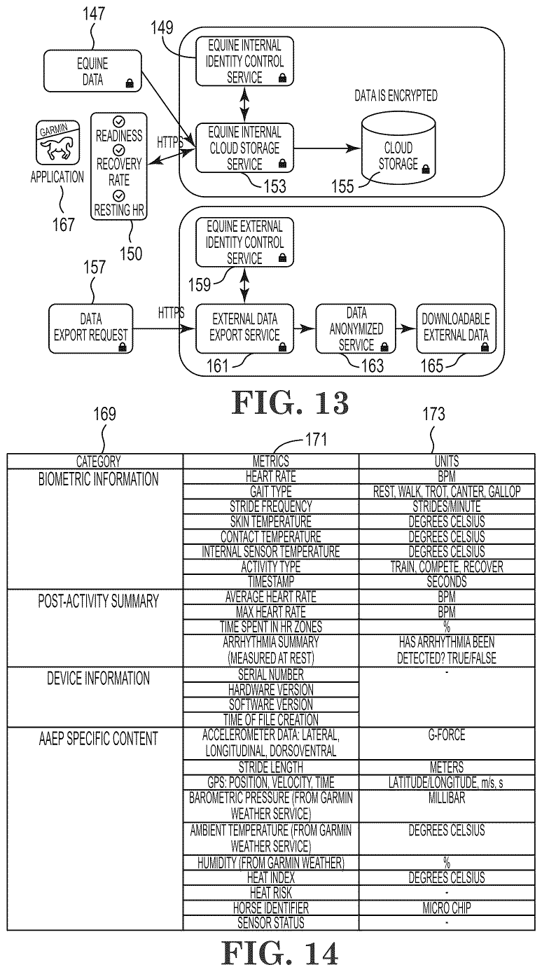

FIG. 13 illustrates an example of a flow diagram for storing and downloading equine data 147. An equine sensor system (e.g., equine sensor system 144 of FIGS. 7 and/or 8) may transmit equine data 147 including kinematic data to an application 167 on computing device 150, which can be a smartphone, a tablet, a laptop, or desktop, with options to connect to an equine internal cloud storage service 153 for cloud storage 155 and trend analysis over time. The equine internal identity control service 149 can ensure the received equine data 147 is matched with the correct equine (e.g., equine 102 of FIGS. 1 and/or 9) and stored with other data corresponding with the equine. The equine data 147 can be encrypted when stored in the cloud storage 155.

In a number of embodiments, a data export request 157 can be received at an external data export service 161. The data export request 157 can come from the American Association of Equine Practitioners (AAEP), for example. The equine external identity control service 159 can match the requested data to the correct equine. In some examples, for instance for research or aggregate analytics, the equine identifiers can be anonymized by the data anonymized service 163 and the downloadable external data 165 can be provided.

Users can review the downloadable external data 165 including metrics across sessions, examining trends in stride length and asymmetry index (e.g., asymmetry index 135 of FIG. 12), which may highlight potential issues. Visual data representations, such as performance charts and comparative analyses, can aid riders, trainers, and veterinarians in evaluating the equine's health, observing progress, and identifying deviations from typical gait patterns.

Owners can use the equine sensor system's GPS capability to monitor each equine's current and historical positional data. By accessing the application 167 or a website portal, users may view each equine's real-time location on a digital map, tracking movements within training fields, pastures, or during transport. Integrated geofencing functionality allows alerts if an equine exits set boundaries, with location insights accessible on any connected smartphone, tablet, or computer.

FIG. 14 illustrates a table of example equine data (e.g., equine data 147 of FIG. 13) stored in a file. An equine sensor system (e.g., equine sensor system 144 of FIGS. 7 and/or 8) can record and store equine data for a number of equines (e.g., equine 102 of FIGS. 1 and/or 9) over time, creating a detailed log of stride length, asymmetry index (e.g., asymmetry index 135 of FIG. 12), and stride frequency, which can be accessed through cloud-based platforms for visualization. The equine data can include metrics 171 with their respective units 173 and each metric of the metrics 171 can be sorted into a category 169. This equine data supports session comparisons and trend analysis, potentially allowing users to detect deviations from normal movement patterns and assess fitness levels. Sharing equine data with veterinarians or trainers may facilitate informed care and training adjustments, supporting management of each equine's health and performance.

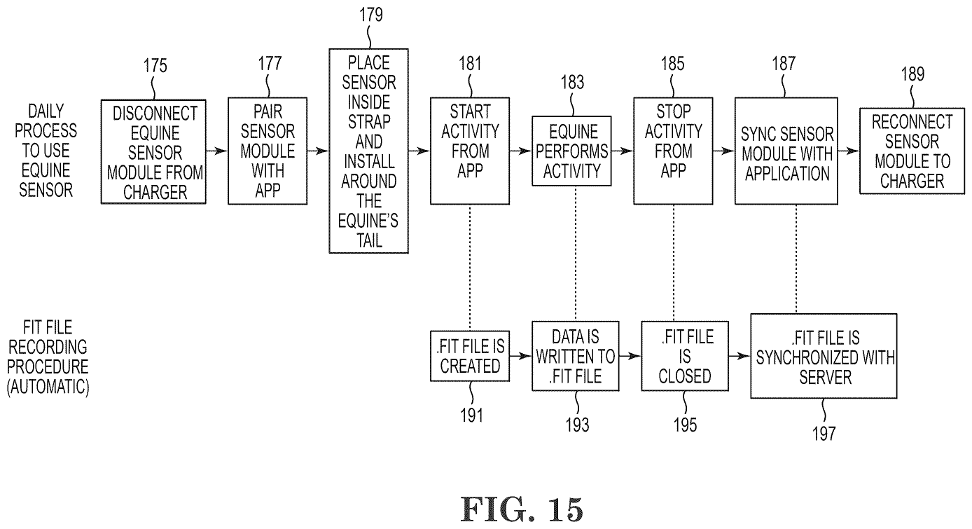

FIG. 15 illustrates an example of a flow diagram for using an equine sensor (e.g., equine sensor 100 of FIGS. 1, 7, and/or 8) and storing equine data (e.g., equine data 147 of FIG. 13). At 175, an equine sensor module (e.g., equine sensor module 121 of FIGS. 1, 2, and/or 3) can be disconnected from a charger. The equine sensor module can be paired with an application (e.g., application 167 of FIG. 13) at 177. At 179, a user can place the equine sensor module inside a strap (e.g., strap 106 of FIG. 1) and install the strap around a tail (e.g., tail 104 of FIG. 1) of an equine (e.g., equine 102 of FIGS. 1 and/or 9). The user can start an activity from the application at 181. At 183, the equine can perform the activity. When the activity is complete, the user can stop the activity from the application at 185. At 187, the equine sensor module can be synced with the application. The user can then reconnect the equine sensor module to the charger at 189.

The equine data collected by the equine sensor module can be formatted and stored in a .FIT file, a widely supported file format for fitness and health metrics, allowing for streamlined data transport and integration across various platforms. For example, at 191, a .FIT file can be created when the user starts the activity from the application at 181. When the equine performs the activity at 183, the equine data can be written to the .FIT file at 193 and when the user stops the activity from the application at 185, the .FIT file is closed at 195. At 197, the .FIT file can be synchronized with a server when the equine sensor module is synced with the application at 187.

By storing equine data including kinematic metrics like stride length, asymmetry index, stride frequency, accelerometer data, and positioning data in a. FIT file, the equine data becomes compatible with multiple analysis tools and software commonly used in athletic performance and veterinary applications. This compatibility enables easy transfer of session data to different devices or software environments for detailed analysis, trend monitoring, or performance review by trainers, veterinarians, or data analysts. The standardized .FIT format also ensures that key metrics are accessible in a consistent structure, supporting interoperability with existing systems that already process similar data types for other fitness activities.

Although specific embodiments have been illustrated and described herein, those of ordinary skill in the art will appreciate that an arrangement calculated to achieve the same results can be substituted for the specific embodiments shown. This disclosure is intended to cover adaptations or variations of one or more embodiments of the present disclosure. It is to be understood that the above description has been made in an illustrative fashion, and not a restrictive one. Combination of the above embodiments, and other embodiments not specifically described herein will be apparent to those of skill in the art upon reviewing the above description. The scope of the one or more embodiments of the present disclosure includes other applications in which the above structures and methods are used. Therefore, the scope of one or more embodiments of the present disclosure should be determined with reference to the appended claims, along with the full range of equivalents to which such claims are entitled.

As used herein, “a number of” something can refer to one or more of such things. As will be appreciated, elements shown in the various embodiments herein can be added, exchanged, and/or eliminated so as to provide a number of additional embodiments of the present disclosure.

In the foregoing Detailed Description, some features are grouped together in a single embodiment for the purpose of streamlining the disclosure. This method of disclosure is not to be interpreted as reflecting an intention that the disclosed embodiments of the present disclosure have to use more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment.

What is claimed is:

1. An equine sensor system, comprising:
an equine sensor comprising:
a strap removably coupled to a tail of an equine;
an accelerometer configured to collect accelerometer data; and
a transmitter coupled to the accelerometer configured to transmit the accelerometer data;
a computing device comprising:
a memory;
a receiver configured to receive the accelerometer data; and
a processor coupled to the memory and the receiver, wherein the processor is configured to determine a stride length of the equine based on the accelerometer data and positional data.

2. The equine sensor system of claim 1, wherein the accelerometer data includes acceleration magnitudes in a dorsal-ventral direction of the equine.

3. The equine sensor system of claim 2, wherein the processor is configured to determine stride frequency of the equine based on the acceleration magnitudes in the dorsal-ventral direction of the equine.

4. The equine sensor system of claim 3, wherein the processor is configured to characterize stride and stance phases of the equine based on the stride frequency of the equine.

5. The equine sensor system of claim 3, wherein the processor is configured to determine a distance traveled by the equine based on the positional data.

6. The equine sensor system of claim 5, wherein the processor is configured to determine the stride length of the equine by dividing the distance traveled by the equine by a number of strides taken by the equine based on the stride frequency of the equine.

7. The equine sensor system of claim 1, wherein the processor is configured to determine a stride consistency metric of the equine based on a number of stride lengths of the equine throughout a work session.

8. The equine sensor system of claim 1, wherein the computing device is a smartphone, a tablet, a laptop, a desktop computer, or a cloud computing device.

9. An equine sensor system, comprising:
an equine sensor comprising:
a strap removably coupled to a tail of an equine;
an accelerometer configured to collect accelerometer data; and
an equine sensor transmitter coupled to the accelerometer configured to transmit the accelerometer data;
a positioning device comprising:
a global navigation satellite system (GNSS) receiver configured to collect positional data; and
a positioning device transmitter coupled to the GNSS receiver configured to transmit the positional data; and
a computing device comprising:
a memory;
a receiver configured to receive the accelerometer data and the positional data; and
a processor coupled to the memory and the receiver, wherein the processor is configured to determine a stride length of the equine based on the accelerometer data and the positional data.

10. The equine sensor system of claim 9, wherein the positioning device is removably coupled to a rider of the equine, clothing of the rider, or equipment of the rider.

11. The equine sensor system of claim 10, wherein the positioning device is a smartwatch.

12. The equine sensor system of claim 9, wherein the positioning device is removably coupled to the equine or tack of the equine.

13. The equine sensor system of claim 9, wherein the accelerometer data includes acceleration magnitudes in a lateral direction, a longitudinal direction, and a dorsal-ventral direction of the equine.

14. An equine sensor system, comprising:
an equine sensor comprising:
a strap removably coupled to a tail of an equine;
an accelerometer configured to collect accelerometer data; and
an equine sensor transmitter coupled to the accelerometer configured to transmit the accelerometer data;
a transponder removably coupled to a rider of the equine, the equine, or the strap, wherein the transponder is configured to transmit a unique signal;
a detection loop configured to receive and transmit the unique signal in response to the transponder passing the detection loop; and
a computing device comprising:
a memory;
a receiver configured to receive the accelerometer data and the unique signal; and
a processor coupled to the memory and the receiver, wherein the processor is configured to determine a stride length of the equine based on the accelerometer data and the unique signal.

15. The equine sensor system of claim 14, wherein the detection loop is an antenna.

16. The equine sensor system of claim 14, wherein the processor is configured to determine a time the transponder passes the detection loop based on the unique signal.

17. The equine sensor system of claim 16, wherein the processor is configured to determine positional data based on the time the transponder passes the detection loop.

18. The equine sensor system of claim 17, wherein the processor is configured to determine the stride length of the equine based on the positional data and the accelerometer data.

19. The equine sensor system of claim 14, further comprising an additional detection loop configured to receive and transmit the unique signal in response to the transponder passing the additional detection loop.

20. The equine sensor system of claim 14, further comprising an additional transponder removably coupled to a different equine or a different rider of the different equine, wherein the additional transponder is configured to transmit a different unique signal.
